(19) United States
(12) Patent Application Publication
(10) Pub. No.: US 2026/0104576 A1
(43) Pub. Date: Apr. 16, 2026

(54) ADAPTIVE OPTICAL CORRECTION IN TWO-PHOTON FLUORESCENCE MICROSCOPY WITH NEURAL FIELDS

(71) Applicant: THE REGENTS OF THE UNIVERSITY OF CALIFORNIA, Oakland, CA (US)

(72) Inventors: Iksung Kang, Berkeley, CA (US); Na Ji, Berkeley, CA (US)

(21) Appl. No.: 19/358,888

(22) Filed: Oct. 15, 2025

Related U.S. Application Data
(60) Provisional application No. 63/707,628, filed on Oct. 15, 2024.

(51) Int. Cl. G02B 21/00; A61B 5/00; G06T 3/02; G06T 5/50; G06T 5/80

(52) U.S. Cl. CPC G02B 21/008; A61B 5/0071; G02B 21/0076; G06T 3/02; G06T 5/50; G06T 5/80; G06T 2207/10056; G06T 2207/10064; G06T 2207/20081; G06T 2207/20084

(57) ABSTRACT

A microscopy system includes a point-scanning fluorescence microscope to acquire a stack of images of an object, an excitation laser arranged to provide laser excitation to the microscope, and one or more processors configured to execute code to cause the one or more processors to acquire a three-dimensional (3D) input image stack of an object using a microscope in the microscopy system, apply neural fields to the 3D input image stack to generate a representation of a sample structure, use an image formation model for the microscopy system to estimate aberration, adjust aberration coefficients to estimate a total aberration based upon the estimated aberration to produce a final aberration correction, and combine the sample structure with the aberration estimation to output an output image stack that matches the 3D input image stack.

### CROSS-REFERENCE TO RELATED APPLICATIONS

This disclosure is a non-provisional of and claims benefit from U.S. Provisional Application No. 63/707,628, titled “ADAPTIVE OPTICAL CORRECTION IN TWO-PHOTON FLUORESCENCE MICROSCOPY,” filed on Oct. 15, 2024, the disclosure of which is incorporated herein by reference in its entirety.

### STATEMENT REGARDING FEDERALLY SPONSORED RESEARCH AND DEVELOPMENT

This invention was made with government support under Grant Number NS118300 awarded by the National Institutes of Health. The government has certain rights in the invention.

### TECHNICAL FIELD

This disclosure relates to point-scanning fluorescence microscopy using adaptive optics and self-supervised machine learning.

### BACKGROUND

Fluorescence imaging of living biological organisms provides mechanistic insights into their physiology. Two-photon (2P) fluorescence microscopy is an essential tool for live imaging, probing structure, and function at subcellular resolution deep within complex tissues. However, as 2P excitation light propagates through tissue, its wavefront accumulates optical aberrations from refractive index mismatches, leading to reduced fluorescence signal, resolution, and contrast. When these sample-induced aberrations are measured and corrected, the excitation light can form a diffraction-limited focus, increasing fluorescence signal and improving the accuracy of structural and functional characterization.

Adaptive optics (AO) measure aberration and correct it with wavefront-shaping devices, such as deformable mirrors (DM) and liquid-crystal spatial light modulators (SLM). AO methods can be grouped into direct wavefront sensing methods, which use a wavefront sensor for aberration measurement, and indirect wavefront sensing methods, which include approaches utilizing machine learning for wavefront estimation.

Regardless of aberration measurement scheme, AO methods are generally developed for and deployed on custom-built microscopes, in which individual optical components are carefully conjugated and aligned to ensure optimal imaging and correction performance. However, microscopes in a general laboratory setting often have imperfect conjugation and misalignment of optical components, with commercial microscopes additionally suffering from limited access and adjustability of their optical paths. Furthermore, sample motion during in vivo imaging leads to artifacts that degrade aberration measurement accuracy, a problem that can be particularly severe for deep tissue imaging as well as for indirect wavefront sensing methods that utilize serial measurement of images and signals.

### BRIEF DESCRIPTION OF THE DRAWINGS

FIG. 1A-FIG. 1D show an embodiment of Neural fields for Adaptive optical Two-photon fluorescence microscopy (NeAT).

FIG. 2A-FIG. 2E show an embodiment of the NeAT.

FIG. 3A and FIG. 3B show embodiments of a point scanning fluorescence microscopy system.

FIG. 4A-FIG. 4O show performance characterization with direct wavefront sensing (DWS) adaptive optics (AO) in a custom-built 2P microscope.

FIG. 5A-FIG. 5C show NeAT corrections for conjugation errors in a commercial microscope.

FIG. 6A-FIG. 6C show real-time aberration corrections by NeAT for in vivo structural imaging.

FIG. 7A-FIG. 7D show examples of convergence and stability of NeAT.

### DETAILED DESCRIPTION OF THE EMBODIMENTS

The embodiments here provide NeAT, Neural fields for Adaptive optical Two-photon fluorescence microscopy. It utilizes neural fields to represent a sample's three-dimensional (3D) structure and incorporates computational architectures to enhance AO performance for imperfect microscopes and living samples. By incorporating an image-formation model for two-photon fluorescence microscopy that accounts for both aberration and sample motion as a physics prior, NeAT accurately estimates aberration from a single fluorescence image stack without requiring external datasets for training, even in the presence of motion artifacts. NeAT also corrects for conjugation errors in the microscope system, ensuring that the corrective phase pattern displayed on a wavefront-shaping device accurately cancels out aberration after propagation through imperfectly conjugated and misaligned optics. Lastly, NeAT jointly recovers the sample's 3D structure with aberration. In scenarios where additional imaging with aberration correction is not needed, NeAT eliminates the need for corrective devices, further reducing system cost and complexity.

One should note that while NeAT uses two-photon fluorescence microscopy, the system and methods here could also apply to other types of point-scanning fluorescence microscopes and microscopy systems. The include, as examples and without limitation, two-photon microscopy system, a confocal microscopy system, and three-photon fluorescence microscopy system.

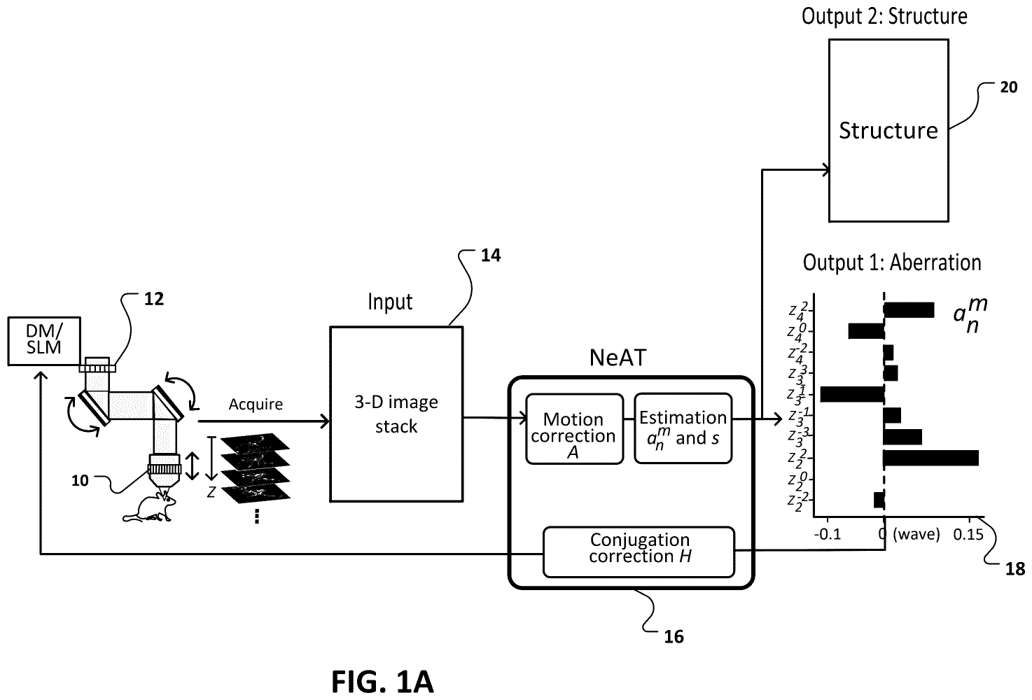

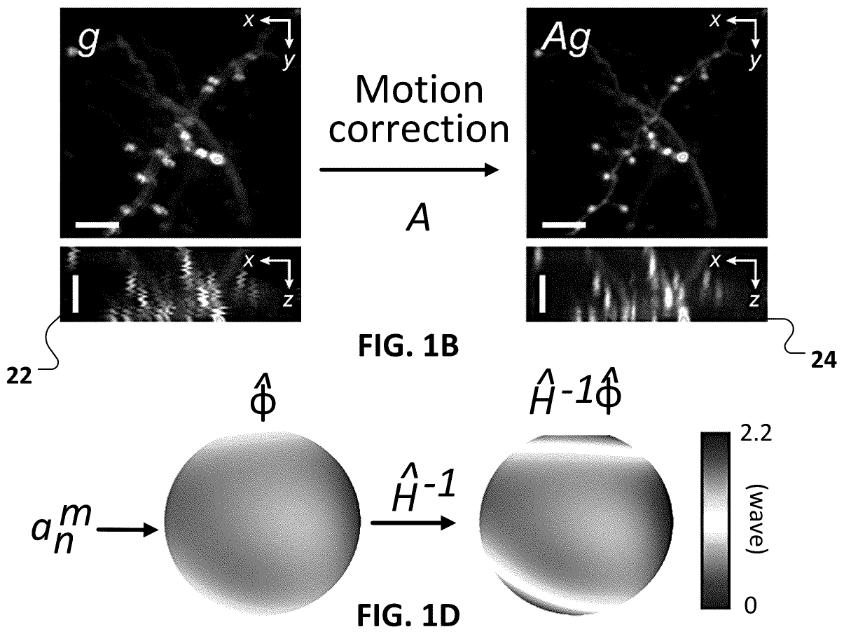

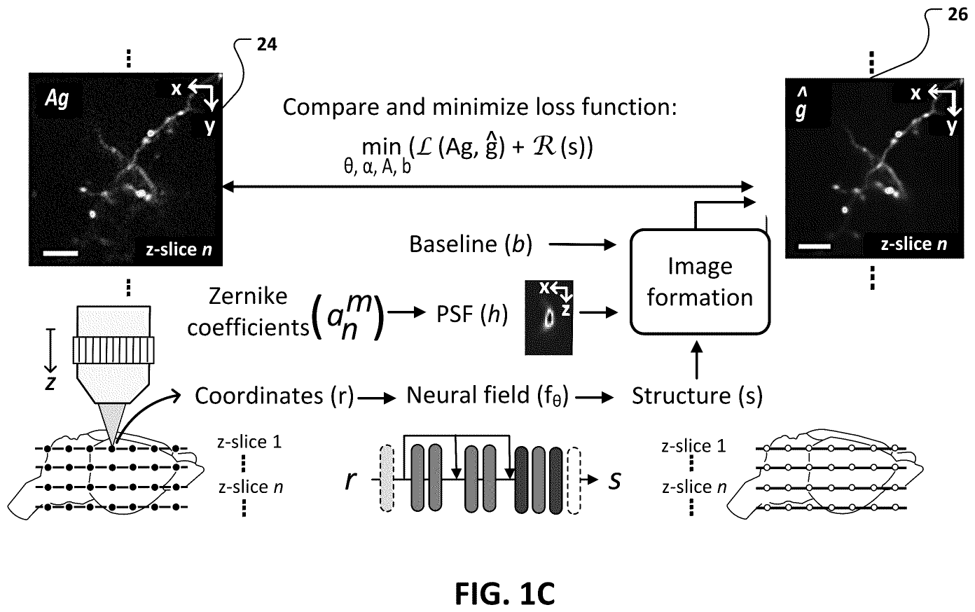

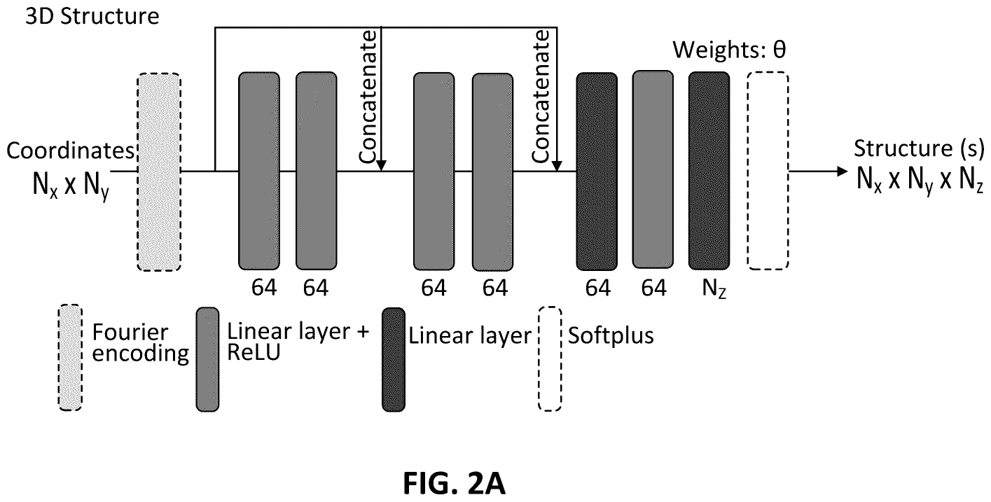

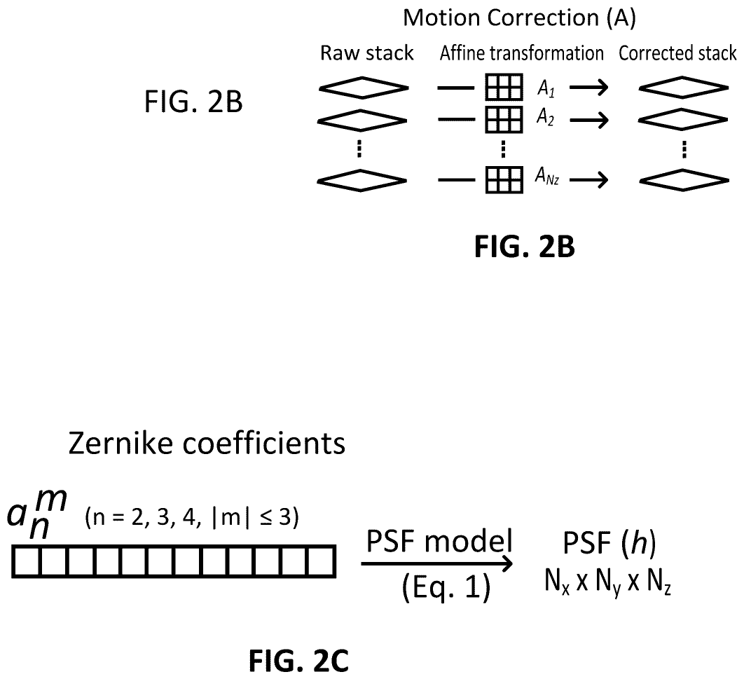

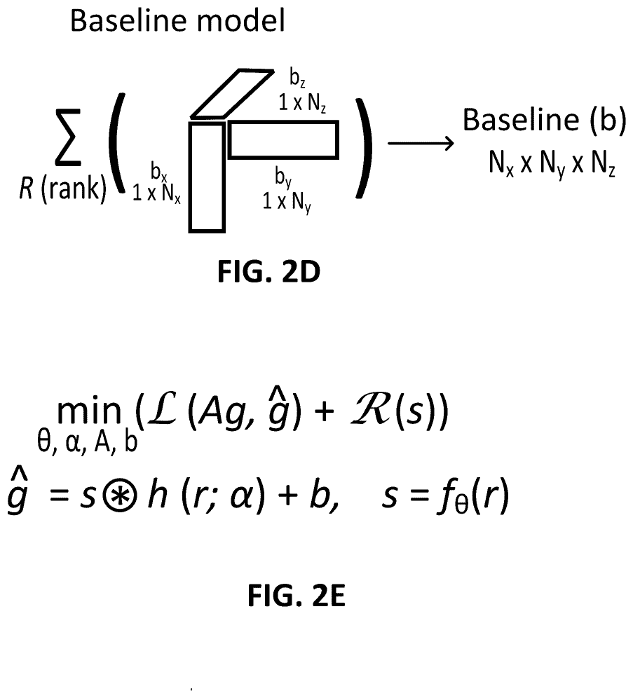

NeAT is designed to jointly estimate wavefront aberration and recover sample structure from an input 3D 2P fluorescence image stack, shown in FIG. 1A. It utilizes neural fields, implicit functions represented by a coordinate-based neural network across spatial coordinates, to represent sample structure. FIG. 2A shows an embodiment of a neural network architecture. NeAT also incorporates a mathematical image-formation model for 2P fluorescence microscopy into the learning process, which involves aberration and structural estimation, as well as motion correction through learnable image transformations. During the learning process, NeAT aims to reproduce an image stack closely resembling the input by iteratively adjusting its parameters. This process requires no external supervision.

The input for NeAT is a three-dimensional (3D) image stack 14 acquired through using a point-scanning fluorescence microscope 10. Artifacts caused by sample motion, such as body movement, breathing, and heartbeat, in the z stack 14, if present, are corrected by a set of affine transformations (A) shown in FIG. 2B, whose parameters are optimized during the learning process shown in FIG. 1A, resulting in an input image 22 in FIG. 1B being corrected to output image 24. In the absence of motion artifacts, A is set as an identity operator and excluded from the learnable parameters.

The image-formation model consists of three components: point spread function (PSF, h), structure(s), and baseline (b), shown in FIG. 1C. The PSF h is computed as:

$$h ( r ; α ) = {\vert F \{ P ( u , v ) {e}^{i φ ( u , v ; α )} {e}^{- 2 π iz \sqrt{{k}_{0}^{2} - {u}^{2} - {v}^{2}}} \} \vert}^{4} . \qquad ( 1 )$$

Here, r represents the spatial coordinates (x, y, z) near the focal plane. P(u, v) and φ(u,v;α) stand for the amplitude and phase maps in the coordinates (u, v) within the circular pupil of the objective lens, respectively. φ(u, v; α) is a linear combination of Zernike modes with their associated coefficients α, with

$$φ ( u , v ; α ) = {\sum }_{\vert m \vert \leq 3} {\sum }_{n = 2}^{4} {α}_{n}^{m} {Z}_{n}^{m} ( u , v ) .$$

Here m and n stand for the angular meridional frequency and radial order, respectively, following the American National Standards Institute (ANSI) standard convention for Zernike modes. α, a 1D tensor, is a set of learned Zernike coefficients shown in FIG. 2C. The aberration estimation was constrained to up to fourth-order Zernike modes, excluding tip, tilt, defocus, and quadrafoil (i.e.,

$${Z}_{n}^{m}$$

with 2≤n≤4 and |m|≤3), based on prior studies. Tip, tilt, and defocus do not affect 2PFM image quality. Quadrafoil is excluded as its inclusion often yields inaccurate estimations under low-signal in vivo imaging conditions.

The 3D structure s, shown in FIG. 1C, is rendered by a neural field shown in FIG. 2A. It takes the spatial coordinates r as input and involves both Fourier-domain spatial encoding and a multi-layer perceptron. This formulation follows the original neural field framework, where spatial coordinates are mapped to radial Fourier features

$$( i . e . , γ ( r ) = {[ sin ( {2}^{l} {R}_{θ} r ) , cos ( {2}^{l} {R}_{θ} r ) ]}_{l = 0 , … , L - 1 ,} where$$

$${R}_{θ} = {\{ [ \begin{matrix}cos ( {θ}_{k} ) & - sin ( {θ}_{k} ) \\ sin ( {θ}_{k} ) & cos ( {θ}_{k} )\end{matrix} ] \}}_{k = 1}^{K} ) , r = ( x , y )$$

denotes the spatial coordinates, L controls the maximum radial frequency depth, and K determines the number of angular samples over 2π. The encoded features are then passed through a multilayer perceptron fθ, which represents the underlying signal as a continuous function shown in FIG. 2A. Structure s is parametrized by the network weights θ and expressed as f_{θ}(γ(r)).

The baseline term b(r) is modeled as the multiplication of three 2D tensors that represent baseline elements along each of the x, y, and z axes as shown in FIG. 1C and FIG. 2D. This term accounts for both the offset due to background fluorescence and noise and, if present, signal decrease along the z axis due to scattering and absorption by tissue.

The image-formation model computes an image stack ĝ from PSF h(r;α), structure s=f_{θ}(γ(r)), and baseline b(r) by convolving PSF with structure before summation with baseline:

$$\hat{g} ( r ) = {f}_{θ} ( γ ( r ) ) h ( r ; α ) + b ( r ) . \qquad ( 3 )$$

NeAT then compares the input stack 12, or more generally with motion correction, Ag of image 24 of FIG. 1C, and the computed stack ĝ image 26. It runs an optimization process to update the learnable parameters over iterations to minimize the loss function as shown in FIG. 1C and FIG. 2E.:

$${min}_{θ , α , A , b} ( ℒ ( Ag , \hat{g} ) + ℛ ( s ) ) . \qquad ( 3 )$$

The fidelity term [symbol] (Ag, ĝ) is a weighted sum of SSIM (Structural Similarity Index Metric) and rMSE (relative Mean-Squared Error) between the two stacks. SSIM evaluates similarity between Ag and ĝ and has been widely used as both an image quality metric and a loss function in computational imaging. The rMSE term computes a weighted L2 loss that reduces the influence of bright pixels and places greater emphasis on minimizing errors in dark regions. The regularization term [symbol] (s) incorporates a generic prior on the spatial piecewise smoothness of the structure and is the summation of three regularizations based on second-order total variation, L1, and nonlinear diffusion. Second-order total variation and L1 regularizations are chosen for rendering spatially sparse structural features (e.g., sparsely labeled neurons). Nonlinear diffusion regularization is employed to avoid both low-frequency and high-frequency artifacts in the structure recovered by NeAT. More detailed information about the image-formation model, loss function, regularization, and two-step learning is discussed in more detail below.

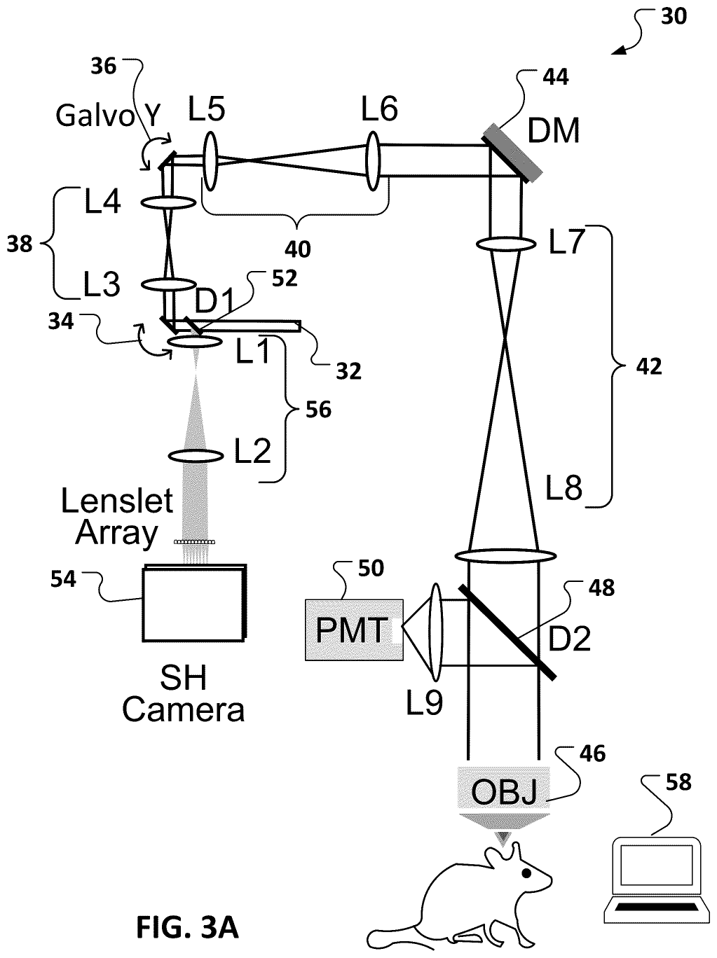

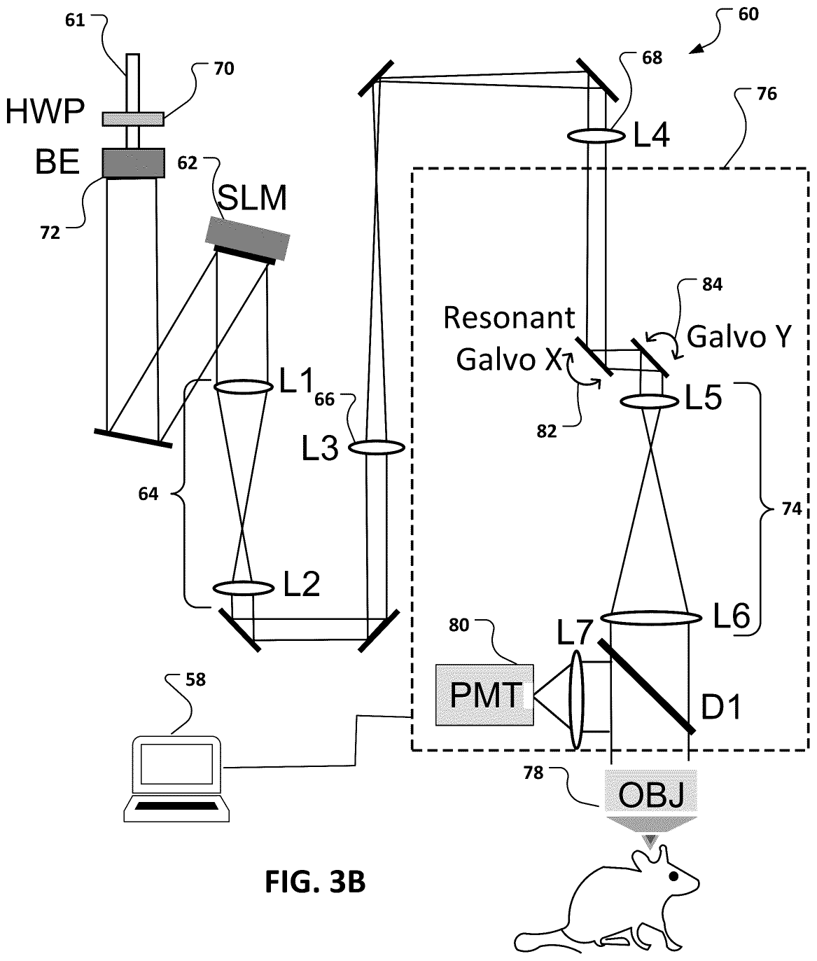

To evaluate the accuracy of NeAT's aberration estimation, the aberration output by NeAT was compared with the ground-truth aberration from DWS with a Shack-Hartmann wavefront sensor of fluorescence from 2P-excited guide stars, using a custom-built 2P microscope with perfect conjugation between optics, including between the X and Y galvos shown in FIG. 3A. Microscope system aberration was measured with DWS and corrected by a DM prior to all experiments.

FIG. 3A shows an embodiment of the custom-built 2P microscope. This merely provides an example of a 2P microscope with perfect conjugation and is not intended to limit application of the embodiments to any particular architecture or components. A custom-built two-photon fluorescence microscope was equipped with a wavefront sensor for DWS. A laser 32 was scanned by a pair of carefully conjugated galvos 34 and 36. Pairs of achromatic doublet lenses, L3-L4 38, L5-L6 40, and L7-L8 42 conjugated the surfaces of galvos with a deformable mirror (DM) 44 and the back focal plane of an objective lens 46. During imaging, 2P excited fluorescence was collected by the same objective lens 46, reflected by a dichroic mirror D2 48, and detected by a photomultiplier tube 50. For wavefront sensing, the emitted 2P fluorescence was descanned by the galvo pair 34, 36, reflected by a dichroic mirror D1 52, and directed to a Shack-Hartmann (SH) sensor 54 through a pair of achromatic lenses L1-L2 56. The SH sensor 54 may comprise a lenslet array conjugated to the objective back focal plane and a CMOS camera positioned at the focal plane of the lenslet array. While most point scanning fluorescence microscopes contain a data acquisition system, not shown, that includes a processor, the implementation of NeAT will typically involve a separate computing device, such as 58. However, as systems develop, the integration of one or more processors into the microscope itself is a possibility. In either case, the system includes one or more processors configured to execute code to implement the embodiments disclosed herein.

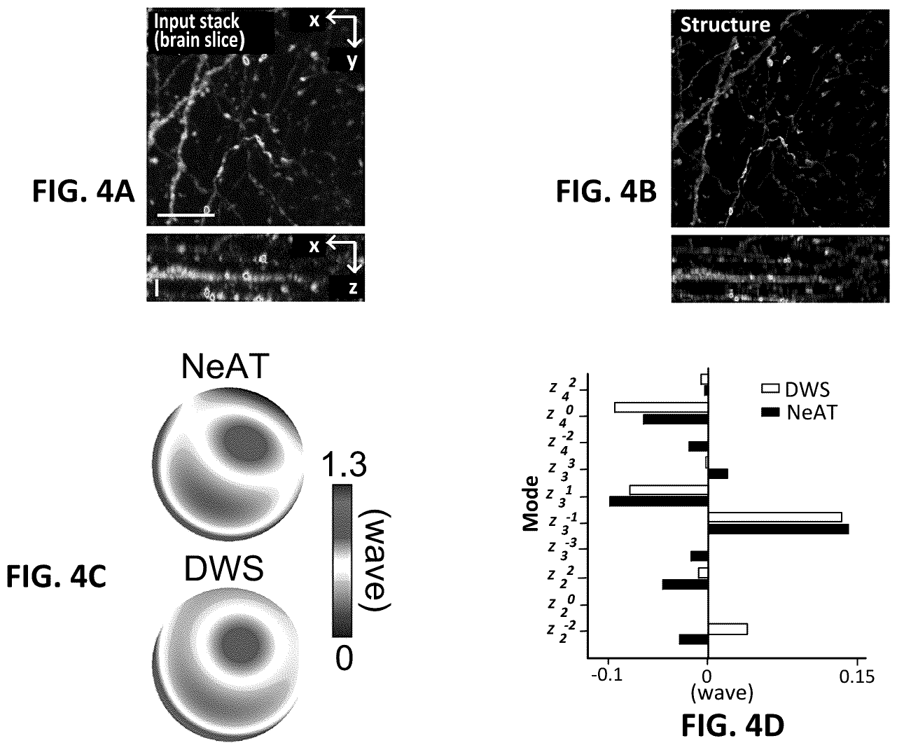

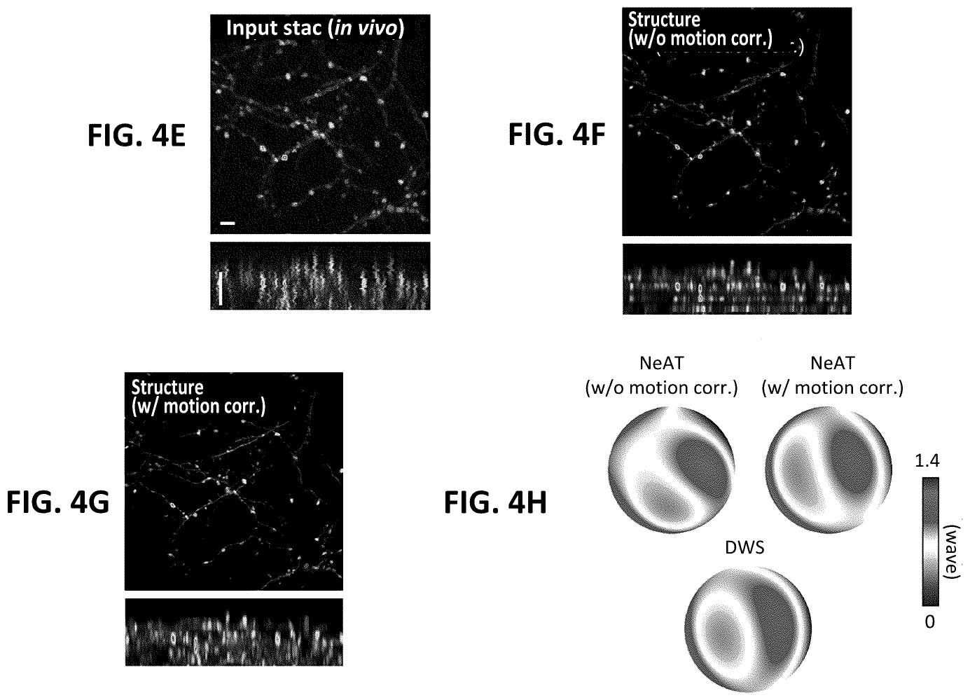

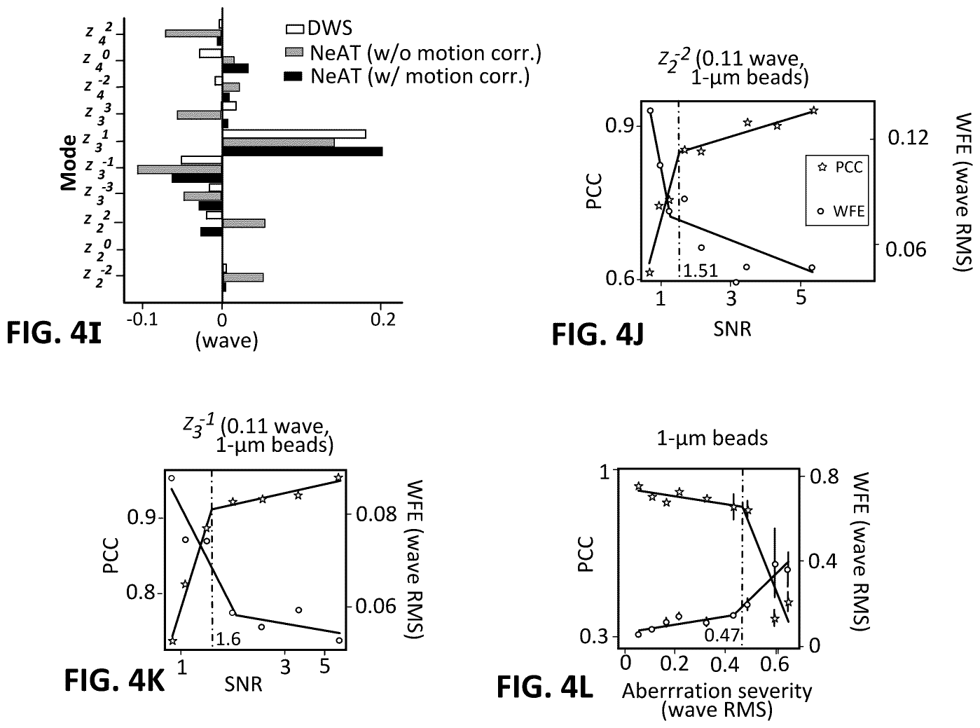

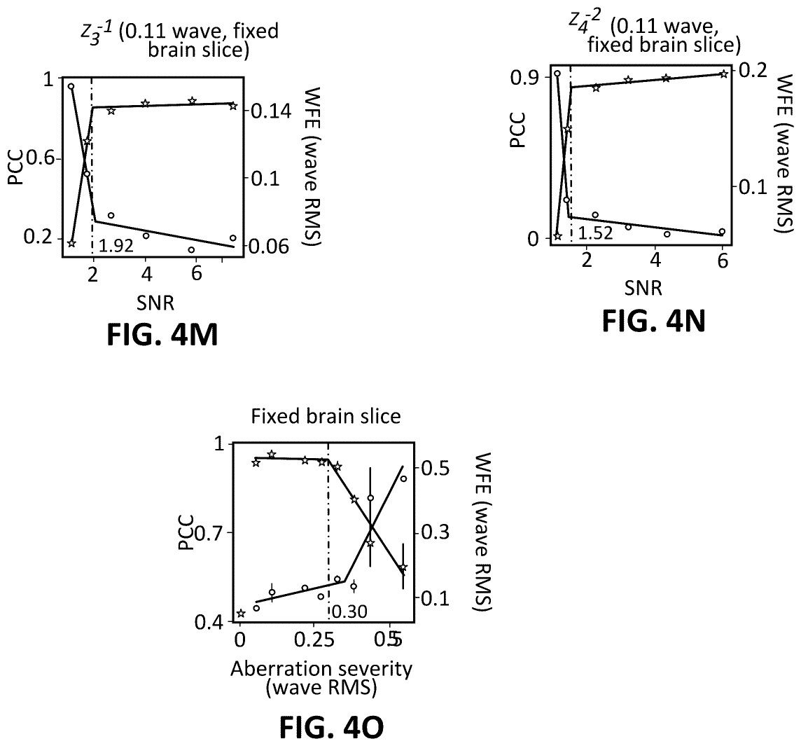

The inventors first validated NeAT's performance using 2P imaging of fixed Thy1-GFP line M mouse brain slices. A #1.5 coverslip was placed above a brain slice at a 3° tilt, which introduced aberrations similar to those typically induced by a cranial window during in vivo mouse brain imaging. The correction collar of the objective lens was set to 0.17, the nominal thickness of the coverslip. From an input image stack of FIG. 4A, NeAT output 3D neuronal structures whose lateral (xy) and axial (xz) maximal intensity projections (MIPs) showed neuronal processes as well as synaptic structures such as boutons and dendritic spines shown in FIG. 4B. The estimated aberration had a similar phase map to the DWS measurement with a root mean square (RMS) difference of 0.09 wave shown in FIG. 4C and comparable coefficients in the dominant aberration modes, e.g., primary coma

$${Z}_{3}^{\pm 1}$$

and spherical

$${Z}_{4}^{0}$$

shown in FIG. 4D. Additional performance validation with DWS showed that NeAT produces aberration estimation comparable to DWS measurement, with RMS differences of less than ˜0.1 waves for both beads and brain slices.

Next, NeAT was applied to in vivo 2P imaging of the mouse cortex. In one mouse, breathing caused lateral shifts between images at different z, show in FIG. 4E. Without correcting for sample motion, the algorithm misinterpreted the laterally displaced images of the same structure at different z as separate structures, leading to striated appearance in the axial MIP of its structural output, shown in FIG. 4F. NeAT addressed this by using affine transformations A to register the image stack, with the transformation matrices jointly learned alongside other parameters, shown in Eq. 3. With sample motion corrected, the structural output was free of striation artifacts as shown in FIG. 4G, and the aberration output much more closely resembled the ground truth have a Root-Mean-Squared (RMS) error of 0.07 wave than the output without motion correction of an RMS error of 0.16 wave, shown in FIGS. 4H and 4I.

The effectiveness of sample motion correction depends on the SNR of fluorescence images as discussed below. For high SNR images (e.g., SNR of 12), NeAT could handle sample motions of ±1 μm of maximum displacement. For noisier images (e.g., SNR of 3), its accuracy decreased and could only handle sample motions with ±0.25 μm displacement. This finding offers practical guidance for optimizing surgical preparation or controlling anesthesia level to minimize sample motion during image acquisition for AO, particularly during deep tissue imaging when SNR is low.

After validating NeAT's performance both in vitro and in vivo, how robustly it performed at varying SNR levels was evaluated. Post-objective power was varied and image stacks were acquired of 1-μm-diameter fluorescence beads at different SNRs with primary astigmatism

$$( {Z}_{2}^{- 2} )$$

or primary coma

$$( {Z}_{3}^{- 1} )$$

introduced to the DM. At low SNR levels (e.g., SNR<1.5), fluorescent beads were poorly visualized and the structures output by NeAT appeared fragmented as they were fitted to noise. Only at sufficiently high SNRs did the structure resemble beads. NeAT's performance was quantitatively evaluated to identify the cutoff SNR below which NeAT's performance deteriorated abruptly. The Pearson correlation coefficient (PCC) was computed between the recovered structures at different SNRs and that from an image stack acquired with no aberration and high SNR, in one example SNR>7. By fitting the PCC values to a piecewise linear curve with two distinct slopes, one can identify the cutoff SNR as 1.51 for astigmatism, shown in FIG. 4J, and 1.60 for coma, shown in FIG. 4K. Below the cutoff SNRs, the accuracy of structural recovery decreased, as indicated by an abrupt drop of PCC values, shown by the stars in in FIG. 4J through 4O. The accuracy of aberration estimation also decreased, as indicated by an increase in wavefront error (WFE), quantified by the RMS error between NeAT's estimate and ground-truth aberrations shown as the circles in FIG. 4J through 4O.

The experiment was repeated on a fixed Thy1-GFP line M mouse brain slice to determine whether similar limits applied to spatially extended biological structures. In this case, primary coma,

$${Z}_{3}^{- 1} ,$$

and secondary astigmatism

$${Z}_{4}^{- 2} ,$$

were applied to the DIVI separately. Similarly to beads, low-SNR images were associated with structures dominated by artifacts. As before, the PCC was calculated between the recovered structures at different SNRs and the ground truth from an image stack acquired with no aberration and high SNR, SNR>5. It was found that the cutoff SNR was 1.92 for coma, shown in FIG. 4M, and 1.52 for astigmatism, shown in FIG. 4N, similar to the cutoff SNRs from the bead data. This suggests that at sufficiently high SNRs (SNR ≳3 for aberrations tested here), NeAT achieves accurate structural recovery, independent of feature characteristics.

Moreover, NeAT's performance limit was characterized in terms of aberration severity. Zernike coefficients were randomly generated to obtain mixed-mode aberrations with RMS values ranging from 0.05 to 0.65 waves. Each aberration was then applied to the DM and acquired images of beads and brain slices at SNR>8. With the increase in aberration, fluorescence images became more degraded in resolution and contrast. At the largest aberrations tested (e.g., 0.65 waves for beads and 0.43 waves for brain slices), the recovered structures no longer accurately represented the features of the beads or neurons.

The PCC was computed between the structures retrieved by NeAT from images with varying levels of external aberration and the structure from an image stack without aberration. Similar to above, the process defined the cutoff RMS as the value above which the PCC exhibited a sudden drop, as identified by fitting the PCC values to a piecewise linear curve with two distinct slopes. A cutoff RMS of 0.47 wave for 1-μm beads in FIG. 4L and 0.30 wave for the brain slice in FIG. 4O was found, respectively. This difference in cutoff RMS values is expected as 3D extended structures generally pose greater challenges than beads.

Lastly, NeAT's performance limit was characterized in terms of sampling rate by varying the pixel sizes of input image stacks. Both in vitro and in vivo image stacks of neurons were downsampled by different factors to vary the input pixel size along the lateral (dx, dy) and axial (dz) axes and compared NeAT's performance in structural recovery and aberration estimation. When pixel size exceeded the Nyquist sampling criterion, the structure outputs from NeAT became inaccurate. The aberration estimation also deviated from the ground truth measured by DWS, with the estimated aberration matching the DWS measurement until lateral pixel size exceeded 0.20 μm and axial pixel size exceeded 0.67 μm, values dictated by the Nyquist condition, for both in vitro and in vivo.

Having demonstrated the successful application of NeAT in a custom-built 2P fluorescence microscope and acquired a thorough understanding of its performance in relation to SNR, motion, aberration severity, and input pixel size, the inventors next tested whether NeAT worked on a commercial 2P microscope. This was motivated by the desire to expand the application of AO beyond optical specialists to a general laboratory setting with microscopes having imperfect conjugation and misalignment of optical components, as well as limited access and adjustability of their optical paths. As with the custom system, this merely provides an example of a commercially-available photon microscope and is not intended to limit application of the embodiments to any particular architecture or components

FIG. 3B shows an embodiment of a commercially available multiphoton microscope 60. A laser 61 is used for 2P excitation. An AO module consisted of a liquid crystal SLM and two pairs of relay lenses, L1-L2 64, and the pair comprised of L3 66 and L4 68 was added to the beam path on the optical table between the laser and the microscope. In one embodiment, the laser output had its polarization rotated by an achromatic half-wave plate 70 to align with the SLM polarization requirement and was expanded using one or more beam expanders represented by beam expander 72 to fill the active area of the SLM 62. The two pairs of relay lenses 64 and 66/68 demagnified the laser output and conjugated the SLM surface to the non-resonant galvo surface within the galvo-resonant-galvo scanning head of the microscope. A pair of scan lenses 74 within the microscope 76 relayed the laser to the back focal plane of a water-dipping objective lens 78. Fluorescence emission was collected through the objective and detected by two photomultiplier tubes 80 for two-color imaging of green using a 525/50 nm emission filter and red 607/70 nm emission filter (not shown) fluorescence, respectively.

A liquid-crystal SLM was added to the beam path between an excitation laser and a commercial 2P fluorescence microscope shown in FIG. 3B. This system differs from the custom-built microscope in several ways. First, the DM, x galvo, and y galvo of the custom-built system were conjugated with pairs of lenses in FIG. 3A to ensure that the corrective phase pattern displayed on the DM was accurately relayed to the back focal plane (BFP) of the objective lens and stayed stationary during beam scanning. But the commercial microscope, as typical for microscopes in biological laboratories, did not conjugate the two galvos 82 and 84 but placed them close to each other. Second, while the optics of the custom-built system were carefully arranged and aligned to ensure the registration between the x and y axes of the SLM surface and the fluorescence images, the commercial microscope had multiple mirrors in an enclosed optical path whose placement and alignment were preset and not adjustable. Finally, the commercial system was designed to have the whole microscope body move in 3D to accommodate large samples, which causes alignment errors between the SLM on the optical table and the objective lens in the microscope that for heavily shared microscopes can vary daily. As a result, a wavefront applied to SLM is translated, rotated, scaled, and/or sheared at the BFP of the objective lens of the commercial microscope, which in turn degrades the performance of aberration correction.

To address this problem, a process to estimate and correct conjugation errors of commercial microscopes was incorporated into the NeAT process as shown in FIG. 1A. Corrective wavefront displayed on the SLM, φ_{corr}, becomes φ_{BFP} at the BFP of the objective lens, with

$${φ}_{BFP} = H {φ}_{Corr} + {Φ}_{Sys} \qquad ( 4 )$$

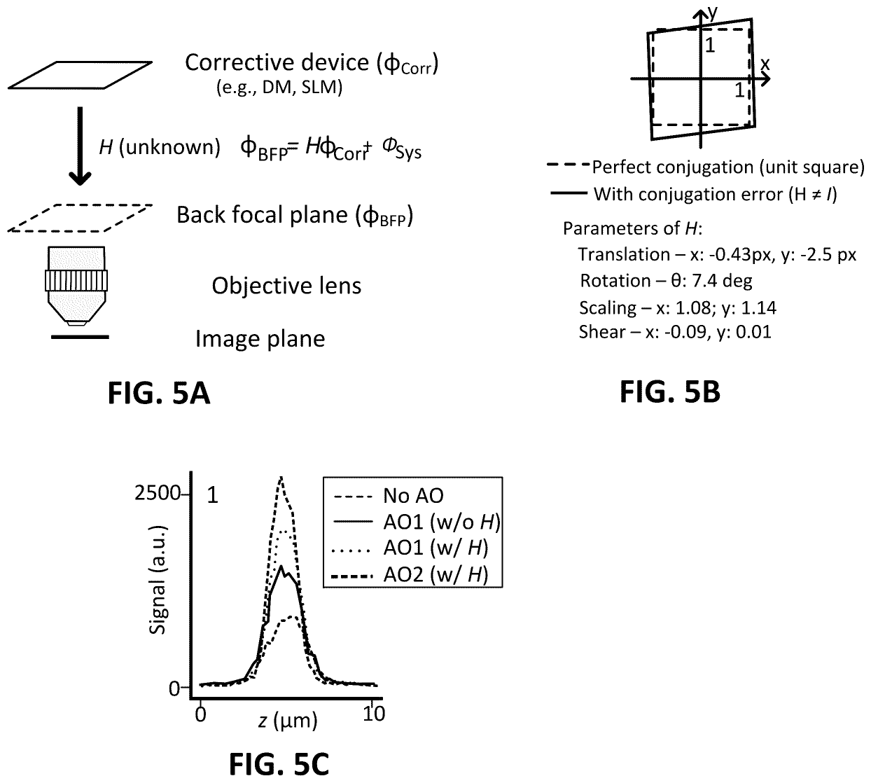

Here Φ_{sys} represents the system aberration and His a linear geometric transformation describing the effects of conjugation errors on φ_{Corr} shown in FIG. 5A. One can model H as an affine transformation with parameters for translational, rotational, scaling, and shear transformation shown in FIG. 5B. For microscopes with perfect conjugation, H=I, the identity operator (i.e., translations are 0 pixels in x and y, rotation is 0 deg, scaling is 1, and shear is 0). For microscopes with conjugation errors, the process of accounting for them requires finding the transformation H and system aberration Φ_{Sys}.

The system aberration Φ_{Sys} was determined by inputting into NeAT an image stack of 200-nm-diameter fluorescence beads acquired with a flat phase pattern applied to the SLM. The estimated system aberration from NeAT is \hat{φ}₀, with

$${Φ}_{Sys} = {\hat{ϕ}}_{0} + {ε}_{0} \qquad ( 5 )$$

Here ε₀ represents estimation error by NeAT, which should be much smaller in RMS magnitude than Φ_{Sys}.

To determine H, one can apply 5 calibration aberrations Φₙ (n=1 to 5) including primary astigmatism

$$( {Z}_{2}^{- 2} and {Z}_{2}^{2} ) ,$$

coma

$$( {Z}_{3}^{- 1} and {Z}_{3}^{1} ) ,$$

and spherical aberration

$$( {Z}_{4}^{0} ) ,$$

to the SLM. These calibration aberrations allow the system to detect translation, scaling, rotation, and shear errors in conjugation. At the objective lens BFP, these aberrations became HΦₙ+Φ_{Sys}. With image stacks of 200-nm fluorescence beads acquired under these external aberrations as inputs shown in FIG. 5C, NeAT returns Øn (n=1 to 5), with

$$H {Φ}_{n} + {Φ}_{Sys} = {\hat{ϕ}}_{n} + {ε}_{n} \qquad ( 6 )$$

Here εₙ represents estimation error by NeAT. Subtracting (5) from (6) and assuming εₙ−ε₀≈0, results in

$$H {Φ}_{n} ≅ {\hat{ϕ}}_{n} - {\hat{ϕ}}_{0} , n = 1 , 2 , … , 5 \qquad ( 7 )$$

Now with Φₙ (n=1 to 5) known, and \hat{φ}ₙ and \hat{φ}₀ from NeAT, one determines the parameters of H by minimizing the loss function:

$$\hat{H} = {arg min}_{H} ( {\sum }_{n = 1}^{5} \vert H {Φ}_{n} - ( {\hat{ϕ}}_{n} - {\hat{ϕ}}_{0} ) \vert ) \qquad ( 8 )$$

Ĥ, the estimate for H, describes how the conjugation errors in the system translate, rotate, scale, and shear the wavefront pattern applied to the SLM on its way to the BFP of the objective lens. To correct these errors, the inverse of Ĥ, Ĥ⁻¹ is applied to the aberration estimation \hat{φ} from NeAT and use Ĥ⁻¹\hat{φ} as the corrective pattern on the SLM as shown in FIG. 1D.

For example, to correct for system aberration of the commercial microscope, an image stack of 200-nm fluorescence beads was used as input to NeAT, which returned \hat{φ}₀ as the aberration estimation. Directly applying \hat{φ}₀ to the SM increased the signal of a fluorescent bead by 1.7-fold. By also correcting for conjugation errors, Ĥ⁻¹ \hat{φ}₀ increased the signal by 2.2-fold. Using the image stack acquired with Ĥ⁻¹\hat{φ}₀ as input into NeAT, the residual aberration

$${\hat{ϕ}}_{0}^{′}$$

was obtained and applied

$${\hat{H}}^{- 1} ( {\hat{ϕ}}_{0} + {\hat{ϕ}}_{0}^{′} )$$

to the SLM, leading to a 3.0-fold signal gain over no aberration correction as shown in the FIG. 5C. From the image stacks acquired with these corrective patterns, NeAT estimated the residual aberrations. Consistent with the fluorescent signal measurements, conjugation error correction substantially reduced residual aberration, with 0.14 and 0.12 wave RMS after the first and second iterations of AO correction, while the residual aberration without conjugation correction had a 0.22 wave RMS.

The approach was further tested on correcting known astigmatism, coma, and spherical aberrations introduced to the SLM. From bead image stacks acquired with these aberrations applied, NeAT returned estimated aberrations, which represented the wavefront distortion at the objective BPF and substantially differed from the applied aberrations due to conjugation errors. Transforming the estimated aberration with Ĥ⁻¹, aberrations were obtained with phase maps that closely matched the given aberrations in all three cases, leading to much smaller RMS errors (astigmatism: 0.087 and 0.19 wave RMS with and without H correction; coma: 0.14 and 0.19 wave RMS with and without H correction; spherical: 0.16 and 0.23 wave RMS with and without H correction). Once characterized, the same Ĥ⁻¹ can be applied as long as the conjugation of the microscope remains unchanged. Below, the system aberration of the commercial microscope was always corrected for “No AO” images so that improvement by AO arose from the correction of sample-induced aberrations alone.

NeAT's capacity to improve was evaluated in vivo structural imaging with the commercial microscope. An image stack of a tdTomato-expressing dendrite were obtained at 350 μm depth in the primary visual cortex (V1) of a head-fixed mouse, and used it as input to NeAT to the SLM the corrective wavefront from NeAT with both motion and conjugation corrections, the same dendrite was imaged and observed a marked improvement in brightness (up to 1.8× for dendritic spines), resolution, and contrast.

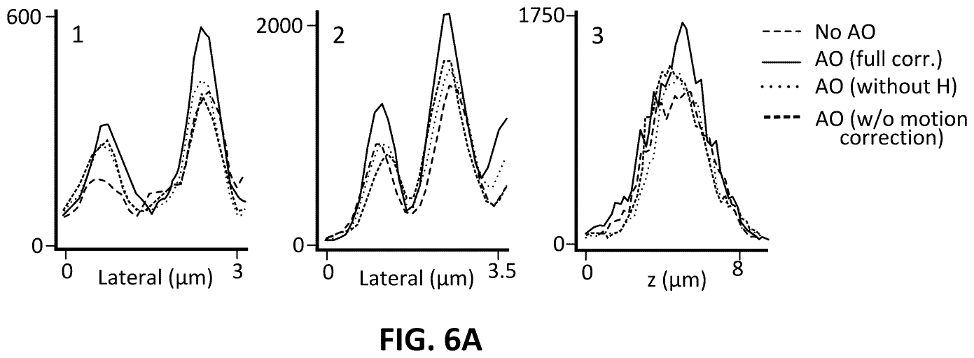

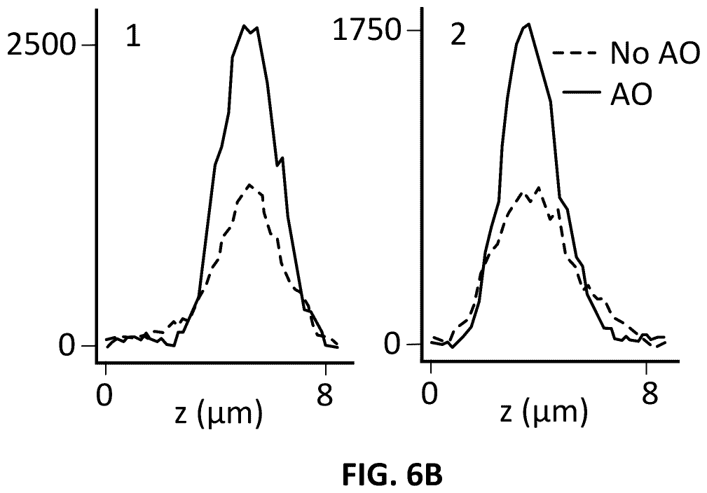

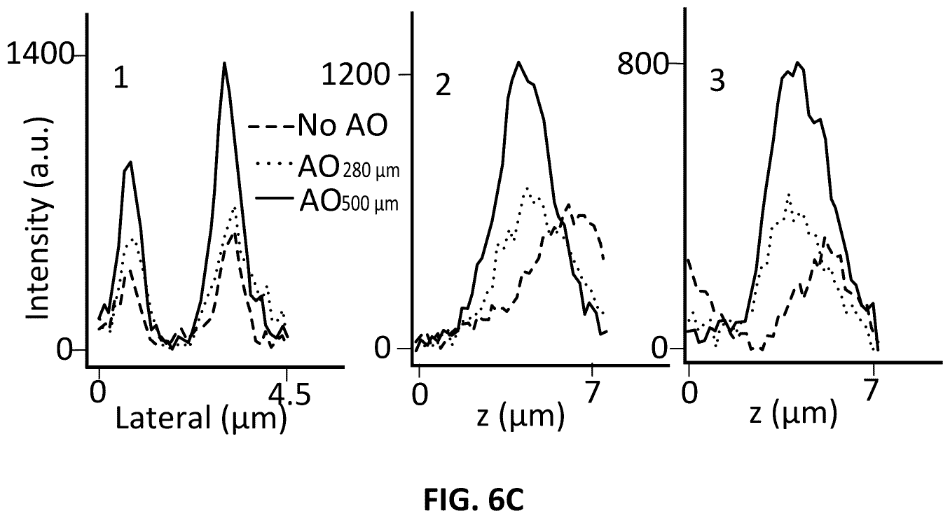

Correcting for both sample motion and conjugation error was necessary for the observed improvement. The corrective wavefront with only motion correction but not conjugation correction substantially differed from that with full correction and led to more modest improvements in image quality. Only correcting for conjugation but not motion similarly underperformed. These trends were observed quantitatively in the lateral and axial intensity profiles of three example dendritic spines shown in FIG. 6A.

The inventors investigated further whether image-registration software such as the StackReg plugin in ImageJ can work similarly well to the motion correction method integrated into the learning process of NeAT. An image stack of beads with aberration was acquired, introduced simulated motion artifacts, pre-registered the stack with StackReg, and then used the resulting image stack as input to NeAT. Although structural recovery was moderately successful for beads, the accuracy of aberration estimation from the pre-registered image stack was inferior to inputting the un-registered stack to NeAT directly. Similarly, pre-registration with StackReg on an image stack acquired in vivo led to a corrective wavefront with smaller brightness improvement than the corrective wavefront from motion correction by NeAT. This can be explained by whether motion correction considers the existence of aberration. While NeAT integrates motion correction into its learning process for aberration (Eq. 3), conventional image registration is unaware of aberrations and matches features between adjacent image planes to align them, which may inadvertently reduce or exaggerate certain aberrations (e.g., the axially curved tail of comatic aberration may be straightened by StackReg).

Having established that both conjugation and motion corrections are needed for in vivo imaging using the commercial microscope, NeAT's performance was further tested for morphological imaging deep within the brain of a Thy1-GFP line M mouse. An image stack acquired at a depth of 280 μm was first used as input to NeAT to obtain the corrective wavefront, 0.36 wave RMS, which led to resolution improvement as well as an ˜2× increase in spine brightness shown in FIG. 6B, where AO substantially enhances the resolution and contrast of fine structures such as dendritic spines. An image stack at 500 μm depth was then acquired while applying to the SLM the corrective wavefront at 280 μm. Using the image stack as input to NeAT, a corrective wavefront was obtained, which was then added to the corrective wavefront at 280 μm to obtain the final corrective pattern at 500 μm depth, 0.49 wave RMS. This corrective wavefront has a larger RMS magnitude than that at 280 μm, consistent with previous observation of stronger aberrations at larger imaging depths for the mouse brain. Compared to the image stacks acquired without AO, the ones with corrective wavefront at 500 μm had the best resolution and contrast, with up to a 2.4-fold increase in brightness for dendritic and synaptic structures shown in FIG. 6C. Here by using the corrective wavefront at a shallower depth when acquiring the input image stack at a deeper depth, it overcame the limit on aberration severity and used NeAT to correct large aberrations experienced in deep tissue imaging.

NeAT was next used with motion and conjugation correction to improve in vivo functional imaging in head-fixed mice. The genetically encoded glutamate indicator iGluSnFR3 was expressed sparsely in V1 neurons. From an image stack of dendrites at 400-μm depth, NeAT returned a corrective wavefront that substantially increased image resolution and contrast, resulting in approximately two-fold improvement in brightness and resolving a dendritic spine from its nearby dendrite.

Subsequently, gratings drifting in eight different directions were presented (0°, 45°, . . . , 315°; 10 repetitions) to the mouse and recorded 2D time-lapse images of dendritic spines in the same FOV at a 60 Hz frame rate, with and without the corrective wavefront applied to the SLM. With iGluSnFR3 labeling, changes in fluorescence brightness reflected glutamate release and thus synaptic input strength at these dendritic spines. Consistent with the above result, AO increased the brightness of dendrites and spines in the averaged time-lapse image. For four representative dendritic spines, ROI 1-4, AO correction doubled the basal intensity (F₀) of their trial-averaged fluorescent traces and led to more prominent glutamate transients with larger amplitudes (Δ/F₀). Fitting the glutamate responses to the 8 drifting grating stimuli with a bimodal Gaussian curve, the orientation-tuning curves for these spines were obtained. Here AO increased the response amplitudes to the preferred grating orientations and led to a higher orientation sensitivity index (OSI) for these spines. Correcting aberration also shifted the preferred orientation of some spines (e.g., ROI 3 and 4), resulting in more similar tuning preference for neighboring spines, consistent with previous findings. Consistently across spine populations, 52 orientation-sensitive ROIs out of 86 spines, aberration correction by NeAT significantly increased basal fluorescence F₀ by 1.9-fold on average (two-sided paired t-test, p<0.001). It also increased V F₀ and OSI values as indicated by pairwise comparison, two-sided paired t-test, p<0.001, and the cumulative OSI distributions (Kolmogorov-Smirnov test, p<0.001.

The inventors further demonstrated that NeAT can also be applied to densely labeled brains, a common application scenario for in vivo calcium imaging of neuronal populations. As NeAT requires an input stack of sparse structures for aberration estimation, viral transduction was used to densely express the genetically encoded calcium indicator GCaMP6s and sparsely express the red fluorescent protein tdTomato in L2/3 neurons of the mouse V1. Because aberration estimation and correction can be performed at different excitation wavelengths without compromising correction performance, an image stack of a tdTomato-expressing neuron was used, acquired with 1000 nm excitation light as the input to NeAT (32×32×10 μm³ stack), to obtain the corrective wavefront. AO visibly improved image contrast and resolution of the tdTomato-expressing neuron, leading to a >2× increase in intensity in both axial profiles at dendritic spines and lateral profiles across dendrites.

Next, the excitation wavelength was switched to 920 nm and 2D images of GCaMP6s-expressing neurons over a 484×484 μm² FOV were obtained, in the green channel at 15 Hz without and with the corrective wavefront obtained by NeAT, while presenting drifting gratings to the head-fixed mouse to evoke calcium responses. The standard deviation images of the time-lapse stacks showed greater intensity differences across time frames after aberration correction, indicative of larger calcium transient magnitude. Indeed, for five representative ROIs, calcium transients were more apparent and had larger magnitudes in both trial-averaged fluorescence (F) and ΔF/F₀ traces with AO, leading to higher orientation selectivity indices for these structures, shown in the right panels.

Over the population of 125 orientation-selective ROIs out of 255 somatic and neuronal structures within the whole FOV, statistically significant differences were found between No AO and AO conditions for both basal fluorescence F₀ (two-sided paired t-test, p<0.001), and ΔF/F₀ (p<0.05). Here the increase in basal fluorescence was less than what was observed for glutamate imaging of dendritic spines, because aberration decreases signal brightness of smaller structures such as dendritic spines more than larger structures such as somata. Similar to glutamate imaging, AO increased the OSIs of neuronal structures, the two-sided paired t-test, p<0.001, and for cumulative distributions of OSI, Kolmogorov-Smirnov test, p<0.001.

The fidelity term [symbol] (Ag, ĝ) in the loss function (Eq. 3) is represented as a weighted sum of SSIM and rMSE as follows,

$$ℒ ( Ag , \hat{g} ) = γ SSIM ( Ag , \hat{g} ) + ( 1 - γ ) rMSE ( Ag , \hat{g} ) , \qquad ( 9 )$$

where rMSE is defined as

$$rMSE ( Ag , \hat{g} ) = {( \frac{Ag - \hat{g}}{sg ( \hat{g} ) + {ε}_{l}} )}^{2} . \qquad ( 10 )$$

Here sg(·) denotes a stop-gradient operation that treats its argument as a constant, employed for numerical stability during backpropagation. This operation prevents the denominator from being differentiated with respect to the network weights, thereby avoiding division-by-zero instabilities, or exploding gradients. The parameter γ controls the weight between the two terms. It is set to 0.25 if the RMS contrast of the image stack's background pixels, i.e., εₗ=σ_{b}(g_{bfr}), is larger than 0.03, where σ_{b}(·) computes the standard deviation of the background pixels of the operand. If the contrast is smaller than 0.02, γ is set to 1.0. Otherwise, γ is linearly interpolated between 0.25 and 1.0. Here, g_{bfr} represents a background-fluctuation-removed version of g, introduced to remove any unwanted low-frequency fluctuations in the images that could otherwise exaggerate the standard deviation.

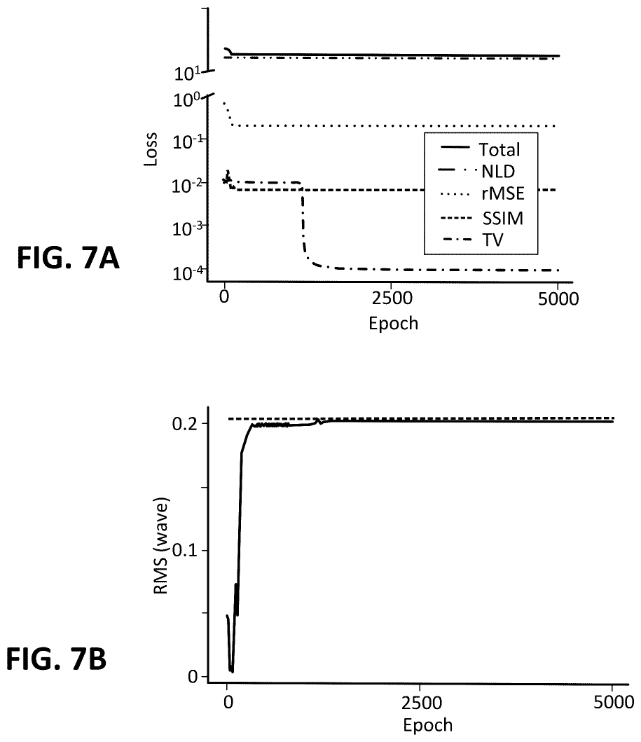

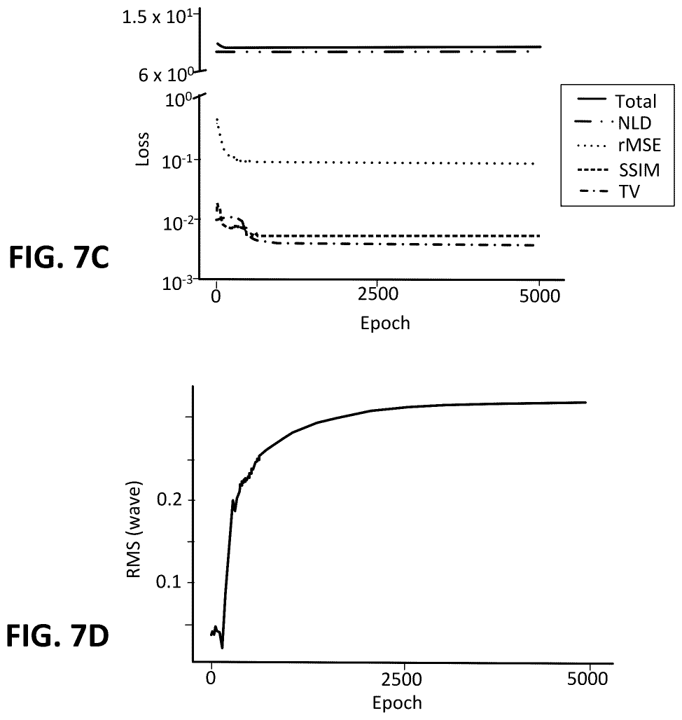

The regularization term [symbol] (s) in the loss function (Eq. 3) is designed to render spatially sparse and smooth structural details, serving as a generic prior that reflects structural features of mouse brain neurons. It includes three regularization terms: second-order total variation (TV) [symbol] ₜᵥ(s), L1 regularization [symbol] _{L1}(s), and nonlinear diffusion (NLD) [symbol] _{NLD}(s). Since the 3D structure s(r)=f_{θ}(γ(r)) is represented implicitly by a neural field, the required spatial derivatives are evaluated directly through automatic differentiation using PyTorch's autograd during the learning process. The convergence behavior of the fidelity and regularization terms remains stable across epochs, same for the aberration estimation, shown for bead samples in FIG. 7A and FIG. 7B, and for brain slices in FIG. 7C and FIG. 7D.

First, second-order TV [symbol] ₜᵥ(s) aims to recover smooth profiles from noisy measurements by sparsifying the spatial gradient components. Unlike first-order TV, which uses first-order derivatives, second-order TV uses second-order derivatives to avoid staircase artifacts. The implementation of the embodiments further applied a nonlinear tone mapping function, an approximated logarithmic function (Eq. 12), which strongly penalizes errors in regions with low intensity values. For simplicity, the spatial coordinates (x, y, z) are expressed as (x₁, x₂, x₃) below.

$$tv ( s ) = {\sum }_{1 \leq i \leq j \leq 3} \vert \frac{{\partial }^{2} s}{\partial {x}_{i} \partial {x}_{j}} \vert , \qquad ( 11 )$$

$${ℛ}_{tv} ( s ) = log ( tv ( s ) + {ε}_{tv} ) ≃ \frac{tv ( s )}{sg ( tv ) + {ε}_{tv}} , \qquad ( 12 )$$

where sg(·) indicates the same stop-gradient operation as above, and εₜᵥ is determined from the input image stack g as the smallest standard deviation of second-order difference tv(g), that is,

$${ε}_{tv} = min ( {σ}_{b} ( \vert \frac{{\partial }^{2} g}{\partial {x}_{i} \partial {x}_{j}} \vert ) ) , 1 \leq i \leq j \leq 3. \qquad ( 13 )$$

Second, L1 regularization [symbol] _{L1}(s) helps to render the structure s with spatially sparse features by adding a penalty based on the absolute value of s as follows,

$${ℛ}_{L 1} ( s ) = log ( \vert s \vert + {ε}_{L 1} ) ≃ \frac{\vert s \vert}{sg ( \vert s \vert ) + {ε}_{L 1}} . \qquad ( 14 )$$

Here ε_{L1}=σ_{b}(|g_{bfr}|) and the same logarithmic tone mapping function (Eq. 12) is applied on the top of the absolute value.

Lastly, NLD regularization [symbol] _{NLD}(s) constrains the magnitude of the first-order difference of the structure s, computed along the depth axis z. This suppresses slowly varying spatial components while preventing the structure from fitting to rapidly varying axial features that sparsity-promoting regularizations might favor (Eqs. 11 and 14). This regularization balances the influence of the first two terms, allowing the structure to retain desirable details. It is written as

$${ℛ}_{NLD} ( s ) = \vert \frac{\partial s}{\partial z} \vert {\vert}_{δ , [ a , b ]} , \qquad ( 15 )$$

where f|_{δ, [a,b]}≡max (f,b)+δ max (a, min(f,b))+min(f,a). For all results presented in this manuscript, δ=0.1, a=0.005, b=2.0.

Together, the summation of the regularization terms is expressed as

$$ℛ ( s ) = {λ}_{tv} {ℛ}_{tv} ( s ) + {λ}_{L 1} {ℛ}_{L 1} ( s ) + {λ}_{NLD} {ℛ}_{NLD} ( s ) , \qquad ( 16 )$$

$$where {λ}_{tv} = 0.005 , {λ}_{L 1} = 0.01 , {λ}_{N L D} = 1 {0}^{- 6} .$$

The baseline term b is modeled as low rank to account for the offset due to baseline fluorescence or noise and potential power decrease along the depth axis caused by scattering in deep tissue imaging. b is represented as the sum of rank-1 tensors, with rank R less than the number of pixels along x, y, and z-axes, Nₓ, N_{y}, N_{z} and set to R=5 here:

$$b = {\sum }_{r = 1}^{R} {b}_{z , r} \times {b}_{y , r} \times {b}_{x , r} , \qquad ( 17 )$$

where b_{x,r}, b_{y,r}, b_{z,r} are learnable 2D tensors to represent baseline components along the x, y, and z axes, respectively. These tensors are initialized with the value (0.1 σ_{b}(|g_{bfr}|))^{1/3}. By constraining b to low rank, it limits it to low-spatial-frequency features, effectively separating background fluorescence from fluorescent features of interest. For input stack g acquired from a mouse brain slice with GFP-expressing neurons, and in vivo from a mouse brain with iGluSnFR3-expressing neurons, the fluorescence baseline is much dimmer than labeled neurons and its low-rank nature is obvious. For the brain slice sample, autofluorescence only exists within the tissue slice and decreases close to zero outside the tissue.

The weights of the neural network θ representing structure s, Zernike coefficients α (thus PSF h(r; α)), and baseline term b in the image-formation model are optimized in a two-step learning process. The first step only adjusts neural network weights for s, while the Zernike coefficients α and baseline b remain fixed after initialization. It conditions the randomly initialized neural network, using the loss function:

$${θ}^{*} = {argmin}_{θ} ( SSIM ( {cg}_{lp} , {f}_{θ} ( r ) ) ) , c \geq 1 \qquad ( 18 )$$

where gₗₚ is a low pass filtered image stack with an isotropic Gaussian filter. Optimization is performed using the RAdam optimizer with an initial learning rate of 10⁻², β₁=0.9, and β₂=0.999 for 5000 epochs. The learning rate schedule follows an exponential decay down to 10⁻³ by the end of the epoch.

The second step updates neural network weights θ, Zernike coefficients α, and baseline b using the loss function (Eq. 3). For this learning process, the initial learning rate is set to 4×10⁻³ with the same RAdam optimizer, keeping β₁ and β₂ unchanged, running for 5000 epochs. The learning rate schedule again follows an exponential decay, this time down to 10⁻⁶ by the end of the epoch.

All computational implementations were performed on a machine equipped with an NVIDIA RTX 4090 GPU, an Intel i9-13900K CPU, and 80 GB of RAM. The estimation times for the results in the main figures are detailed in Table 1, along with their corresponding experimental settings. Overall, the computational cost of NeAT scales approximately linearly with the square root of total number of pixels in the XY images of the input stack, while remaining constant with respect to the axial depth (for a fixed number of z-slices).

TABLE 1
Experimental Settings and estimation time of NeAT

| | Thy1- | Thy1- | | | | | | |
|---|---|---|---|---|---|---|---|---|
| | GFP | GFP | 200-nm | | Thy1- | Thy1- | | C57BL/6J |
| | line M | line M | fluores- | C57BL/6J | GFP | GFP | C57BL/6J | in vivo |
| Sample | brain | in | cence | in vivo | line M | line M | in vivo | (tdTomato, |
| type | slice | vivo | beads | (tdTomato) | in vivo | in vivo | (iGluSnFR3) | GCaMP6s) |
| Scope Type | Custom | Custom | Comm. | Comm. | Comm. | Comm. | Comm. | Comm. |
| Excitation | 920 | 920 | 920 | 1000 | 920 | 920 | 920 | 1000 |
| Wavelength | | | | | | | | |
| (nm) | | | | | | | | |
| Post- | 4 | 6 | 4 | 21 | 23 | 50 | 72 | 29 |
| objective | | | | | | | | |
| Power | | | | | | | | |
| (mW) | | | | | | | | |
| Numerical | 1.1 | 1.1 | 1.05 | 1.05 | 1.05 | 1.05 | 1.05 | 1.05 |
| aperture | | | | | | | | |
| Input stack | 34.2 × | 76 × | 25 × | 25 × | 25 × | 25 × | 28 × | 25 × |
| size (μm³) | 34.2 × 20 | 76 × 10 | 25 × 10 | 25 × 10 | 25 × 20 | 25 × 20 | 28 × 8 | 25 × 10 |
| Pixel size | (0.086, | (0.19, | (0.125, | (0.125, | (0.125, | (0.125, | (0.125, | (0.125, |
| (dx, dy, | 0.086, 0.2) | 0.19, 0.2) | 0.125, 0.2) | 0.125, 0.2) | 0.125, 0.4) | 0.125, 0.4) | 0.125, 0.2) | 0.125, 0.2) |
| dz) (μm) | | | | | | | | |
| Motion | No | Yes | No | Yes | Yes | Yes | Yes | Yes |
| correction | | | | | | | | |
| Estimation | 493 | 436 | 83 | 245 | 245 | 245 | 200 | 245 |
| Time (s) | | | | | | | | |

In the experimental settings, the raw 3D experimental fluorescence image stacks have dimensions of N_{f}×N_{z}×N_{y}×Nₓ, where N_{f} denotes the number of frames per z-axis slice, N_{z} the number of z-axis slices, and Nₓ and N_{y} the number of pixels along the x- and y-axes, respectively. Here N_{f} frames are acquired per z-axis slice to reduce the effect of Gaussian noise through averaging. In a typical experiment, N_{f}=50 frames were acquired at a z-axis slice, before advancing to the next z-slice and acquiring another N_{f} frames. Collecting N_{z} (typically 50) z-axis slices in this manner required approximately 1.5 minutes in total. In in vivo imaging experiments, the frames acquired at the same z may need to be registered before averaging to correct for sample motion between frames. The inventors used a customized ImageJ plugin to register the frames for each z-axis slice in 2D using the TurboReg plugin with a rigid body assumption and then to average the N_{f} registered frames to obtain the image stack with dimensions of N_{z}×N_{y}×Nₓ. The resulting stack is cropped to remove edge pixels and then used as input to NeAT. The input stack dimensions listed in Table 1 For the in vivo image stacks, the dimensions are set to 50×200×200.

Typical input image stack extends 10 μm in z, within which aberrations are effectively constant, an assumption supported by observations that applying an aberration correction estimated at one depth substantially improves signal intensity and contrast across adjacent depths spanning at least ˜100 μm (i.e., ±50 μm).

Whether denoising by Noise2Void could reduce frame averaging requirements was explored, but found that at the SNR of the in vivo images, such a computational denoising approach caused errors in aberration estimation when the number of averaged frames was too low. Therefore, for the SNR ranges that were explored, frame averaging remains the preferred approach for noise reduction.

Image stacks acquired in vivo can contain motion artifacts caused by heartbeats, breathing, or body movements. Although preprocessing as described above removes the motion artifacts for frames acquired at the same z depth, motion between frames at different z depths also needs to be corrected to ensure that structural features are properly aligned for accurate aberration estimation by NeAT. Motion correction for the input z-stack therefore refers to registration across different z positions. Failing to do so would lead to errors in aberration estimation and structural recovery, shown in FIG. 6A. NeAT incorporates motion correction across z slices into its learning process by assigning an affine transformation matrix, Aₙ_{_{z}} (n_{z}=1, 2, . . . , N_{z}), to each z slice to correct translation, rotation, scaling, and shear caused by the sample's motion. This is formulated as (Ag)[n_{z}]=Aₙ_{_{z}}g[n_{z}], where g[n_{z}] denotes the n_{z}-th z-slice of the input image stack g. NeAT corrects motion by iteratively updating these matrices during the learning process. Each matrix is a 2×3 matrices with learnable elements and initialized as the identity. The process used the RAdam optimizer for the motion correction process, with an initial learning rate of 0.07, β₁=0.9, and β₂=0.999. Through aberration-aware motion registration, for image stacks with aberration, NeAT leads to different affine parameters from those estimated by StackReg in ImageJ and achieves more accurate aberration measurement than pre-registering the image stack prior to learning using StackReg.

A linear relationship was assumed between the grayscale pixel value (d) and the photon count per pixel (p_{c}), expressed as d=βp_{c}, where β is the conversion factor. To compute β, multiple images were acquired (e.g., more than 100) of a fluorescein solution at the same imaging condition. The variance and mean were calculated for d. Since p_{c} theoretically follows a Poissonian distribution, where the variance equals the mean, β is computed as the ratio of the variance to the mean of d.

For the cutoff SNR analysis, β was calculated for the PMT in the custom-build microscope under different control voltages, observing gains of 7.83 at a control voltage of 0.7 V (used for acquiring images from 1-μm fluorescence beads, FIGS. 4J-L and 21.8 at a control voltage of 0.8 V (used for fixed Thy1-GFP mouse brain slice imaging, FIGS. 4M-O).

Next, the pixels in an image stack were classified as either signal or background pixels using a classification method described previously. The SNR of the image stack was then calculated as

$$SNR = \frac{\overline{y} / β}{\sqrt{\overline{y} / β}} = \sqrt{\overline{y} / β} , \qquad ( 19 )$$

where y̅ is the mean grayscale value of the signal pixels, and y̅/β represents the corresponding photon count. The inventors developed an ImageJ plugin to compute the SNR of a 3D image stack and determine whether it possesses sufficient SNR to be used as NeAT input (i.e., its SNR should exceed the SNR cutoff).

Visual stimuli were generated in MATLAB using the Psychophysics Toolbox and presented 15 cm from the left eye of the mouse on a gamma-corrected, LED-backlit LCD monitor with a mean luminance of 20 cd·m. The monitor was divided into a 3×3 grid and presented 1-s-long uniform flashes in a pseudorandom sequence in one of the 9 grids, while recording fluorescence images with a 2 mm by 2 mm FOV. Analyzing these images allowed identification the cortical region that responded to the center of the monitor. This cortical region was then imaged at smaller pixel sizes to measure glutamate and calcium activity of synapses and neurons towards oriented drifting grating stimulation in mice under light anesthesia (0.5% isoflurane in O₂). Full-field gratings of 100% contrast, a spatial frequency of 0.04 cycles per degree, and a temporal frequency of 2 Hz drifting in eight directions (0° to 315° at 45° increments) were presented in pseudorandom sequences. For glutamate imaging (x and y pixel size: 0.125 μm/pixel), each grating stimulus lasted 2 s with a 1-s presentation of a gray screen before and after the stimulus. For calcium imaging (x and y pixel size: 0.945 μm/pixel), each grating stimulus lasted 2 s with a 1-s gray screen presentation before and a 3-s gray screen presentation after the stimulus. Each stimulus was repeated for 10 trials per imaging session.

Images were processed using custom Python code. Glutamate time-lapse images were registered using iterative phase correlation with polar transform (implemented using the scikit-image Python package) to correct for non-rigid motions including translation, rotation, and scaling. The images from the first trial of visual stimulation (a 4-second-long recording) were used as the reference. Within this reference time series, each frame was registered to the first frame. Then an average was calculated from all registered frames therein and used as the reference. Images from subsequent trials were registered to this reference. Calcium time-lapse images were registered with the StackReg package, with the first frame as the reference. In this case, the polar transform method proved more effective than StackReg for registering glutamate images, which were dimmer and noisier than calcium images. Regions of interest (ROIs) were manually drawn in ImageJ using the circular selection tool on the mean intensity projection of the glutamate time-lapse images and elliptical selection tool for the GCaMP6s time-lapse images. The ROIs were then imported into a Python environment to extract pixel values within the ROIs, which were averaged to obtain the raw fluorescence signal F for each ROI.

The glutamate transient ΔF/F₀ was calculated as (F−F₀)/F₀, where F₀ represents the basal fluorescence, defined as the average fluorescence signal during the 1-s pre-stimulus gray-screen presentation period, excluding the highest 5% of values in/from the calculation.

For calcium images, due to higher labeling density, neuropil contamination was removed. Fₙₑᵤᵣₒₚᵢₗ was calculated as the averaged fluorescence signal from the neuropil area (defined as the pixels that were 2 to 20 pixels off the ROI border) and computed ΔFₙₑᵤᵣₒₚᵢₗ as Fₙₑᵤᵣₒₚᵢₗ−F_{0, neuropil}, where F_{0, neuropil} is the mean of Fₙₑᵤᵣₒₚᵢₗ during the 1-s pre-stimulus period. Then, ΔFₙₑᵤᵣₒₚᵢₗ was multiplied by 0.7 and subtracted from F to obtain Fₜᵣᵤₑ. ΔF/F₀ was then computed as (Fₜᵣᵤₑ−F_{0,true})/F_{0,true}, with F_{0,true} defined as the mean of Fₜᵣᵤₑ during the 1-s pre-stimulus period.

Trial-averaged ΔF/F₀ was calculated as the average of 10 trials. Peak ΔF/F₀ was defined as the maximal trial-averaged ΔF/F₀ within the 2-s drifting grating presentation. Response R for each drifting grating direction was defined as the averaged ΔF/F₀ across the 2-s drifting-grating stimulus presentation, with negative responses set to zero.

For glutamate images, an ROI was considered responsive to visual stimulation if its peak ΔF/F₀ was greater than 3 times the standard deviation of the trial-averaged ΔF/F₀ within the 2-s stimulus period and if the peak ΔF/F₀ was above 5%. For calcium images, an ROI was considered active if its maximal ΔF/F₀ was above 10% and visually responsive if its activity during at least one visual stimulus type was significantly higher than its activity during the pre-stimulus period, as determined by one-way ANOVA with p<0.01.

For each ROI, its tuning curve R_{fit}(θ) was defined as the fitted curve to R(θ) with a bimodal Gaussian function:

$${R}_{fit} ( θ ) = {R}_{0} + {A}_{1} {e}^{- \frac{ang {( θ - {θ}_{pref} )}^{2}}{2 {σ}^{2}}} + {A}_{2} {e}^{- \frac{ang {( θ - {θ}_{pref} + 180 ° )}^{2}}{2 {σ}^{2}}} , \qquad ( 20 )$$

where ang(x)=min(|x|, |x−360°|, |x+360° |), which wraps the angular values onto the interval between 0° and 180°. Responses to the different drifting direction R(θ) were fitted to the function to minimize the mean square error between the model and responses, with R₀, A₁, A₂ constrained to non-negative values, and σ constrained to be larger than 22.5°, given that the angle step was 45°.

ROIs were considered orientation-sensitive (OS) if their responses across 8 different drifting grating stimuli were significantly different by one-way ANOVA (p<0.05) and if their responses were well-fit to the bimodal Gaussian model. The goodness of the fit was assessed by calculating the error E and the coefficient of determination [symbol] ²:

$$E = {\sum }_{n = 0}^{7} {( R ( θ ) - {R}_{fit} ( θ ) )}^{2} {\vert}_{θ = {( 45 n )}^{°}} , {2}^{} = 1 - \frac{E}{{\sum }_{n = 0}^{7} {( R ( θ ) - \overline{R} )}^{2} {\vert}_{θ = {( 45 n )}^{°}}} , \qquad ( 21 )$$

where R̅ is the mean of R(θ). The criteria for a good fit were E<0.01 and [symbol] ²>0.5. The fitted response was used to calculate orientation sensitivity index (OSI) as

$$\frac{{R}_{pref} - {R}_{ortho}}{{R}_{pref} + {R}_{ortho}} ,$$

where R_{pref} and Rₒᵣₜₕₒ are the responses at θ_{pref} and θₒᵣₜₕₒ(=θ_{pref}+90°), respectively.

The embodiments herein describe NeAT, a general-purpose AO framework for aberration measurement and correction for 2P microscopy using neural fields. Neural fields refer to implicit functions represented by a coordinate-based neural network across spatial coordinates. They have been used for various computational imaging applications, including Neural Radiance Fields (NeRF) for 3D scene representation. NeAT has several distinct features that set it apart from NeRF and other neural field applications, detailed in Table 2, below, including its incorporation of a physics-based prior specific to the 2P imaging system, its estimation and correction of sample motion and microscope conjugation errors, and its joint recovery of 3D structural information alongside aberration estimation.

TABLE 2
Differences between NeRF and NeAT

| | NeRF | NeAT |
|---|---|---|
| Input | Set of 2D images from | 3D image stack from a |
| | various viewpoints | single viewpoint |
| Output | Color, density | Zernike coefficients, |
| | | structure |
| Image formation | Ray tracing for 3D | Two-photon fluorescence |
| model | scene reconstruction | microscopy |
| Loss function | Mean square error | Hybrid loss (SSIM, |
| | | relative MSE) |
| Application | Computer graphics, | In vivo imaging with |
| | virtual reality | adaptive optics |
| Additional | | Conjugation error |
| features | | correction, motion |
| | | correction, aberration |
| | | estimation |

Using the physics-based prior of the 2P imaging system, implemented through an image-formation model that accounts for both aberrations and sample motion, NeAT accurately estimates optical aberrations from a 2P fluorescence image stack without the need for external supervision, even in the presence of motion artifacts during live animal imaging. Importantly, this functionality eliminates the need for integrating AO capabilities into microscope control software, making it applicable to existing custom-built and commercial 2P microscopy systems in general.

Furthermore, NeAT can estimate and correct conjugation errors. Such errors are common in homebuilt and commercial microscopes used in general biology laboratories and cause the distortion of the applied corrective phase pattern at the objective lens back focal plane, leading to deteriorated AO performance. NeAT measures the impact of conjugation errors on calibration aberrations and compensates for them by preemptively transforming the corrective phase pattern before it is displayed on the wavefront-shaping device. This feature would greatly facilitate the broader adoption of AO in a wide range of microscope systems.

Additionally, NeAT simultaneously recovers 3D structural information while estimating aberration during its learning process. For applications where only structural information is needed, this unique capability eliminates the requirement of wavefront-shaping devices or the need for additional imaging with AO correction, greatly lowering system complexity and cost.

NeAT's performance was evaluated under various conditions, such as different SNR levels, aberration severity, and motion artifacts. The inventors established the framework's performance limits for accurate and reliable operation, providing valuable guidelines on imaging settings when applying NeAT.

Finally, NeAT was applied to in vivo structural and functional imaging of the mouse brain by a commercial microscope, demonstrating its capability in improving image quality for demanding real-life biological applications. NeAT effectively estimates and corrects aberrations deep into the mouse brain, enabling morphological imaging of synapses at 500 μm with improved resolution and contrast. It also improves the signal and accuracy of glutamate and calcium imaging of synapses and neurons in the mouse visual cortex responding to visual stimuli.

With a single z-stack as input and a computation time of a few minutes shown in Table 1, NeAT's simple implementation, robust performance, and ability to correct for motion and conjugation errors in imaging systems offer great potential for broader adoption and impact in biological research than many hardware-based AO methods.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

Although specific aspects of this disclosure have been illustrated and described for purposes of illustration, it will be understood that various modifications may be made without departing from the spirit and scope of the invention. Accordingly, the invention should not be limited except as by the appended claims.

What is claimed is:

1. A method of correcting images obtained by a point-scanning fluorescence microscopy system, comprising:
acquiring a three-dimensional (3D) input image stack of an object using a microscope in the microscopy system;
applying neural fields to the 3D input image stack to generate a representation of a sample structure;
using an image formation model for the microscopy system to estimate aberration;
adjusting aberration coefficients to estimate a total aberration based upon the estimated aberration to produce a final aberration correction; and
combining the sample structure with the aberration estimation to output an output image stack that matches the 3D input image stack.

2. The method as claimed in claim 1, wherein the point-scanning fluorescence microscopy system includes an optical correction device, and the method further comprises applying the final aberration correction to an optical correction device to improve image quality.

3. The method as claimed in claim 1, wherein acquiring the 3D input image stack of the object comprises acquiring one of morphological images or activity images.

4. The method as claimed in claim 1, wherein using the image formation model comprises:
adjusting Zernike coefficients based upon the 3D input image stack of images to produce the estimated aberration;
combining the estimated aberration with the image formation model to generate a point spread function;
combining the point spread function with the sample structure to compute the output image stack; and
minimizing a difference between the 3D input image stack and the output image stack.

5. The method as claimed in claim 1, further comprising applying a set of affine transformation to correct for motion artifacts in images in the 3D stack of images.

6. The method as claimed in claim 5, wherein the affine transformations are used in a learning process for the image formation model.

7. The method as claimed in claim 1, further comprising correcting conjugation errors in the point-scanning fluorescence microscopy system, comprising:
acquiring a 3D stack of images of an object using the microscope in the microscopy system;
displaying a set of calibration wavefront patterns on an optical correction device and acquiring a calibration stack of images corresponding to the set of calibration wavefront patterns from the microscope;
estimating any conjugation errors detected in the calibration stack of images;
adjusting a corrective wavefront applied to the optical correction device for any conjugation errors based on any estimated conjugation errors found; and
outputting a conjugation-error-corrected wavefront pattern for aberration correction.

8. The method as claimed in claim 7, wherein estimating any conjugation errors comprises:
determining an initial estimated affine transformation from the calibration stack of images; and
minimizing a loss function between the estimated affine transformation and an ideal affine transformation to produce an estimated affine transformation.

9. The method as claimed in claim 8, wherein adjusting the corrective wavefront comprises applying an inverse of the estimated affine transformation to the optical correction device.

10. A microscopy system, comprising:
a point-scanning fluorescence microscope to acquire a stack of images of an object;
an excitation laser arranged to provide laser excitation to the microscope; and
one or more processors configured to execute code to cause the one or more processors to:
acquire a three-dimensional (3D) input image stack of an object using a microscope in the microscopy system;
apply neural fields to the 3D input image stack to generate a representation of a sample structure;
use an image formation model for the microscopy system to estimate aberration;
adjust aberration coefficients to estimate a total aberration based upon the estimated aberration to produce a final aberration correction; and
combine the sample structure with the aberration estimation to output an output image stack that matches the 3D input image stack.

11. The system as claimed in claim 10, further comprising an optical correction device in an optical path between the laser and the microscope, and the one or more processors are further configured to apply the final aberration correction to the optical correction device.

12. The system as claimed in claim 10, wherein the code that causes the one or more processors to use the image formation model comprises code that causes the one or more processors to:
adjust Zernike coefficients based upon the 3D input stack of images to produce the estimated aberration;
combine the estimated aberration with the image formation model to generate a point spread function;
combine the point spread function with the sample structure to compute the output image stack; and
minimize a difference between the 3D input image stack and the output image stack.

13. The system as claimed in claim 10, further comprising applying a set of affine transformation to correct for motion artifacts in images in the 3D stack of images.

14. The system as claimed in claim 13, wherein the affine transformations are used in a learning process for the image formation model.

15. The system as claimed in claim 10, wherein the one or more processors are further configured to execute code that causes the one or more processors to correct conjugation errors in the point-scanning fluorescence microscope by:
acquire a 3D stack of images of an object using the microscope in the microscopy system;
display a set of calibration wavefront patterns on an optical correction device and acquiring a calibration stack of images corresponding to the set of calibration wavefront patterns from the microscope;
estimate any conjugation errors detected in the calibration stack of images;
adjust a corrective wavefront applied to the optical correction device for any conjugation errors based on any estimated conjugation errors found; and
output a conjugation-error-corrected wavefront pattern for aberration correction.

16. The system as claimed in claim 15, wherein the code that causes the one or more processors to estimate any conjugation errors comprises code that causes the one or more processors to:
determine an initial estimated affine transformation from the calibration stack of images; and
minimize a loss function between the estimated affine transformation and an ideal affine transformation to produce an estimated affine transformation.

17. The system as claimed in claim 15, wherein the code that causes the one or more processors to adjust the corrective wavefront comprises code that causes the one or more processors to apply an inverse of an estimated affine transformation to the optical correction device.

18. The system as claimed in claim 10, wherein the point-scanning fluorescence microscope comprises one of a two-photon microscopy system, a confocal microscopy system, and three-photon fluorescence microscopy system.
